(19) United States
(12) Patent Application Publication
(10) Pub. No.: US 2011/0118590 A1
(43) Pub. Date: May 19, 2011

(54) System For Continuous Cardiac Imaging And Mapping

(75) Inventor: Hongxuan Zhang, Palatine, IL (US)

(73) Assignee: SIEMENS MEDICAL SOLUTIONS USA, INC., Malvern, PA (US)

(21) Appl. No.: 12/838,569

(22) Filed: Jul. 19, 2010

Related U.S. Application Data
(60) Provisional application No. 61/262,166, filed on Nov. 18, 2009.

(51) Int. Cl. A61B 5/05

(52) U.S. Cl. 600/424

(57) ABSTRACT

A system improves precision and reliability of intra-cardiac catheter position tracking and monitoring. An interventional system for internal anatomical examination includes a catheterization device for internal anatomical insertion. The catheterization device includes, at least one magnetic field sensor for generating an electrical signal in response to rotational movement of the at least one sensor about an axis through the catheterization device within a magnetic field applied externally to patient anatomy and a signal interface for buffering the electrical signal for further processing. A signal processor processes the buffered electrical signal to derive a signal indicative of angle of rotation of the catheterization device relative to a reference. The angle of rotation is about an axis through the catheterization device. A reproduction device presents a user with data indicating the angle of rotation of the catheterization device.

This is a non-provisional application of provisional application Ser. No. 61/262,166 filed Nov. 18, 2009, by H. Zhang.

### FIELD OF THE INVENTION

This invention concerns an interventional system for internal anatomical examination, by using a catheterization device for internal anatomical insertion and by presenting a user with data indicating the angle of rotation of the catheterization device.

### BACKGROUND OF THE INVENTION

Angiography (or arteriography) imaging is widely used to visualize cardiac chamber size and segmental wall mobility and coronary size, morphology, flow, anatomy and arterial luminal size by displaying static and dynamic image silhouettes. This provides the ability to assess cardiac and coronary arterial function and calculate estimations of chamber volumes (Ventricular and Atrial) to support diagnosis of cardiac disease. Accurate catheter position tracking and location are desirable to capture cardiac electrophysiological activities and tissue functions. Known systems determine catheter position inside the heart using catheter tracking in a Carlo mapping system (e.g., a system provided by a company such as Biosense Webster) and velocity image mapping system (e.g., provided by St. Jude Medical). However it is also desirable to know the degree of catheter rotation and twisting angle for cardiac signal acquisition and image mapping, such as for an intra-cardiac ultrasound catheter which provides real time heart function imaging at certain angles and determines area by using crystal echo methods. Known systems for intra-cardiac catheter manipulation and rotation tracking are typically not accurate and reliable and need extensive expertise and clinical experience for synchronizing catheter rotation with data and image acquisition.

Stable, accurate and high quality image scanning is desirable for analysis and diagnosis of cardiac function and tissue status to identify cardiac diseases and pathology. Known imaging systems, such as X-ray or ultrasound imaging systems, usually capture images arbitrarily while an intra-cardiac catheter is being moved. This means catheter movement and cardiac image acquisition are not synchronized and a physician has to rely on experience to adapt and judge catheter position and image acquisition. This is subjective and prone to error. For example, an intra-cardiac ultrasound catheter is inserted in a heart chamber and uses an oscillating crystal to acquire an ultrasound image with limited echo angle. A user needs to move and rotate the catheter to select a region of interest (ROI) position, depth and direction to obtain the best quality 3D image data. Acquired images need to be synchronized for each catheter position and rotation angle to reconstruct an accurate 3D image and catheter tracking map.

Known systems lack a capability for intra-cardiac catheter rotation and position tracking for image acquisition and interpretation which impedes accurate 3D image construction using 2D scanned images. Furthermore, intra-cardiac catheter steering by manual or motor control involves nonlinear and non-uniform catheter movements. Known catheter tracking systems (such as magnetic coil, X-ray, fMRI systems) fail to accurately track nonlinear movements of EP (electrophysiological) signal catheters, ablation catheters, ultrasound catheters and balloon catheters, for example.

Heart image reconstruction (e.g., in 3D) in known intra-cardiac ultrasound echo image systems is impaired because of lack of 2D image synchronization with catheter spatial position, especially in rotation angle. Further, sensitivity and stability of current 3D image systems used for intra-cardiac applications depend on different factors: such as catheter position, catheter rotation, patient movement and electrical artifacts. The absence of twist and rotation angle tracking in known system renders spatial information indicating catheter location potentially inaccurate and results in distorted 3D image construction. A system according to invention principles addresses these deficiencies and related problems.

### SUMMARY OF THE INVENTION

A system improves precision and reliability of intra-cardiac catheter position tracking and monitoring, especially catheter rotation and twisting using a magnetic sensor and field based intra-cardiac catheter for tracking catheter position (including individual leads in the catheter), XYZ coordinate spatial position and rotation angle. An interventional system for internal anatomical examination includes a catheterization device for internal anatomical insertion. The catheterization device includes, at least one magnetic field sensor for generating an electrical signal in response to rotational movement of the at least one sensor about an axis through the catheterization device within a magnetic field applied externally to patient anatomy and a signal interface for buffering the electrical signal for further processing. A signal processor processes the buffered electrical signal to derive a signal indicative of angle of rotation of the catheterization device relative to a reference. The angle of rotation is about an axis through the catheterization device. A reproduction device presents a user with data indicating the angle of rotation of the catheterization device.

### BRIEF DESCRIPTION OF THE DRAWING

FIG. 1 shows an interventional system for internal anatomical examination, according to invention principles.

FIG. 2 shows a catheter tip, including localization sensors, patient signal transducers and sensors, according to invention principles.

FIG. 3 shows a catheter localization sensor set including 3 coil magnetic sensors, according to invention principles.

FIG. 4 shows a flowchart of a process employed by a catheter system for intra-cardiac patient signal and image acquisition and scanning, according to invention principles.

FIG. 5 illustrates intra-cardiac ultrasound catheter based 3D image scanning and reconstruction including catheter rotation tracking, according to invention principles.

FIG. 6 shows different embodiments of an ablation catheter, according to invention principles.

FIG. 7 shows a flowchart of a process used by an interventional system for internal anatomical examination, according to invention principles.

### DETAILED DESCRIPTION OF THE INVENTION

A system improves precision and reliability of intra-cardiac catheter position tracking and monitoring, including catheter rotation and twisting using magnetic sensors. The system tracks catheter position and individual leads in a multi-lead (e.g., basket) catheter by determining XYZ coordinate spatial position and rotation angle. The system provides continuous heart condition mapping using 3D image acquisition and reconstruction enabled by accurate rotation tracking, localization and guidance of an intra-cardiac catheter. The continuous 3D intra-cardiac system imaging and mapping indicate detailed cardiac function status as well as location and severity of cardiac pathology and clinical events. The system provides a more efficient, accurate and reliable method for evaluating patient health status, identifying cardiac disorders, differentiating cardiac arrhythmias, characterizing pathological severity, predicting life-threatening events, and evaluating the drug delivery and effects.

A system according to invention principles provides continuous cardiac imaging and mapping based on tracking non-uniform catheter rotating angles. The system advantageously detects small position changes and detects position (including focal angle, twisting, XYZ coordinate position and rotation angle) of different portions of an intra-cardiac catheter (or other kinds of catheter, such as an ultrasound catheter or ablation catheter). The system facilitates active catheter medical image scanning and treatment including intra-cardiac ultrasound scanning and catheter direction and angle based ablation, and intra-cardiac medicine delivery. The catheter rotation and twisting tracking and localization system enables 3D image construction of a heart system with high stability and accuracy, e.g. for ultrasonic beam based scanning and image acquisition. Further, image acquisition with limited angle scanning is advantageously compensated with accurate catheter position and rotation tracking and synchronization. The system employs smart sensors to provide real time catheter tracking with improved precision, stability and sensitivity and decreases radioactivity dosage, such as from X-ray scanning for catheter tracking.

The system supports real time 3D monitoring and characterization of heart tissue function and diagnosis of heart rhythm, tissue function, and circulation by detection of small changes in distribution and transition of cardiac arrhythmias. The interventional system enables detection and tracking of position, rotation and twist of individual catheter segments to synchronize image scanning and acquisition with catheter rotation and degree of twist for intra-cardiac ultrasonic beam based image mapping of a heart. The system also supports synchronized 2D and 3D image scanning and construction with improved ablation operation. Intra-cardiac catheters are used for cardiac function analysis. However catheter manipulation depends on clinical user experience and catheter guidance involves nonlinear and non-uniform movement increments. This increases the difficulty of tracking small changes in catheter position, especially of a flexible material catheter that twists and rotates. In one embodiment a catheter, combines a normal catheter lead (patient signal sensor or transducer in treatment delivery equipment) and a position (angle) sensor for use in an electrophysiological catheter as well as other kinds of intra-cardiac equipment, such as an ultrasound catheter, ablation catheter and ICD (implantable cardioverter-defibrillator).

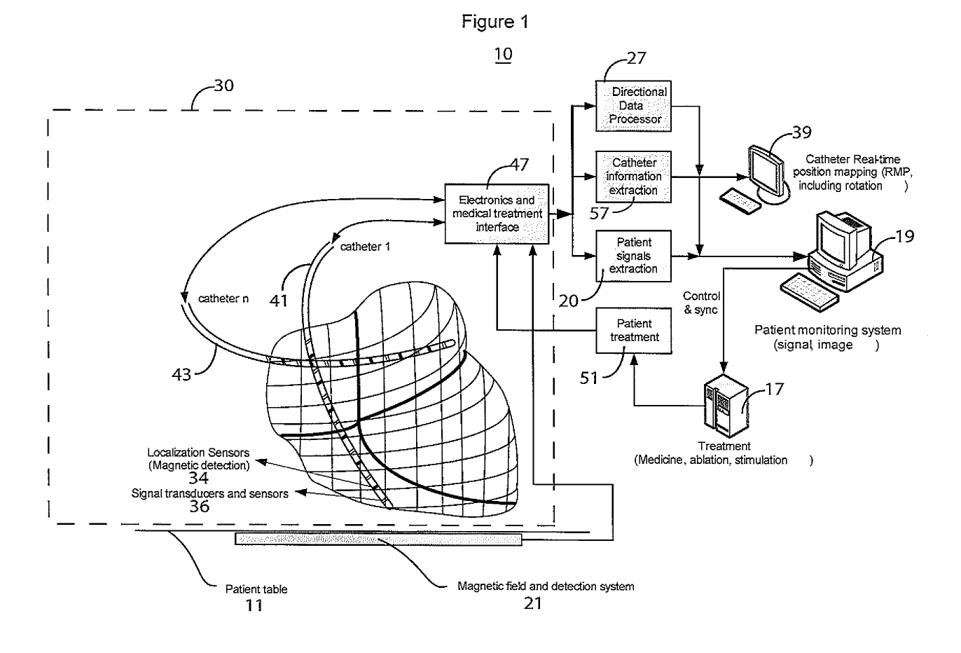

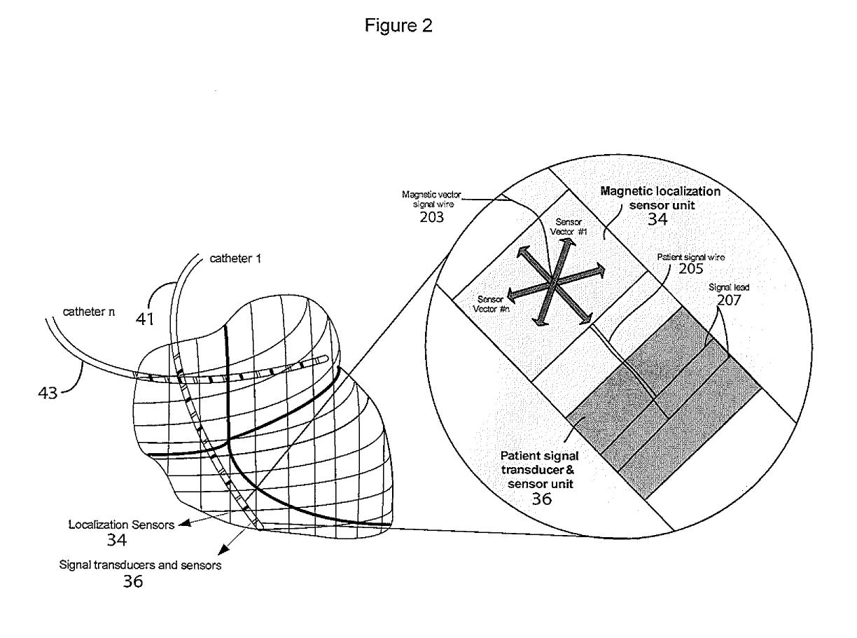

FIG. 1 shows interventional system 10 for internal anatomical examination including catheter system 30 comprising multiple intra-cardiac catheters such as catheters 41 and 43 and signal interface 47. A catheter such as catheter 41 or 43 includes, electrophysiological sensors and transducers 36 for acquiring image data and signals indicating cardiac tissue activities and localization sensors and transducers 34 for providing signals for use in determining catheter position, rotation angle and degree of twist. The localization sensor and a clinical function catheter are combined to form a single catheter (e.g., catheter 41) capable of rotation and twist tracking, signal acquisition and monitoring and treatment (such as by ablation and medicine delivery). In system 10, coil sensor 34 in catheter 41 and magnetic field system 21 (e.g., under patient support table 11) operate together to localize the position, rotation and angle of catheter 41. A tip of catheter 41 includes EP signal sensors 36 and catheter position localization sensors 34 (as described in more detail in connection with FIG. 2 later). The catheter position localization sensor is used for catheter movement tracking based on interaction of a magnetic field and a sensor coil. Catheter movement (such as rotation) is determined by tracking the signal of the position sensor based on copper coil movement within a magnetic field creating a voltage potential used by system 10 to determine the angle and direction of coil sensor movement.

System 10 unit 51 and treatment information database 17 supports different kinds of treatment delivery via catheter 41 including, high-voltage ultrasonic beam stimulation delivered via an intra-cardiac ultrasound catheter, ablation to destroy tissue, drug delivery and electrical stimulation, for example. Ultrasound echo response signals acquired via catheter 41 are converted to electrical signals and extracted using electronics and medical treatment interface 47. The catheter angulation and rotation position is obtained from the parallel magnetic localization sensors 34 within the catheter and are utilized to synchronize signal and image scanning and data acquisition. Interventional system 10 for internal anatomical examination, comprises catheterization device 30 for internal anatomical insertion. Device 30 includes, at least one magnetic field sensor 34 for generating an electrical signal in response to rotational movement of the at least one magnetic field sensor about an axis through the catheterization device within a magnetic field applied externally to patient anatomy by magnetic field system 21. Device 30 includes signal interface 47 for buffering the electrical signal for further processing. Signal processor units 20 and 57 process the buffered electrical signal to derive a signal indicative of angle of rotation of catheterization device 30 relative to a reference and about an axis through catheter 41. Reproduction devices 19 and 39 present a user with data indicating the angle of rotation of catheterization device 30. Directional data processor 27 determines direction in three dimensional space of at least a portion of catheterization device 30 in response to movement of multiple sensors 34 in catheterization device 30 within a magnetic field applied externally to patient anatomy by magnetic field system 21.

FIG. 2 shows an expanded catheter segment or tip, including localization sensors 34 and patient signal transducers and sensors 36 with self-rotation tracking capability. Magnetic localization sensor units 34 in individual segments of catheter 41 are able to track small changes of position of a tip or individual segment of intra-cardiac catheter 41 and track nonlinear rotation of each individual segment of the catheter as well as non-uniform angular changes of the catheter tip or segment and provides 3D XYZ coordinate spatial information to interface 47 via leads 203. Focal position changes (such as of angle and rotation) of different portions of the catheter are tracked using set of magnetic sensors 34 to obtain accurate catheter movement information. The number of sensors in magnetic localization sensor unit 34 is determined by mechanical requirements and clinical accuracy requirements (such as a 0.1-0.5 degree angle differentiation capability requirement). FIG. 2 shows there are 3 sensors in sensor unit 34 and angle between each sensor is 120 degrees in the same plane (cross-sectional plane of catheter 41). Electrophysiological sensors 36 acquire signals from patient tissue via leads 207 and provide the signals to interface 47 via leads 205.

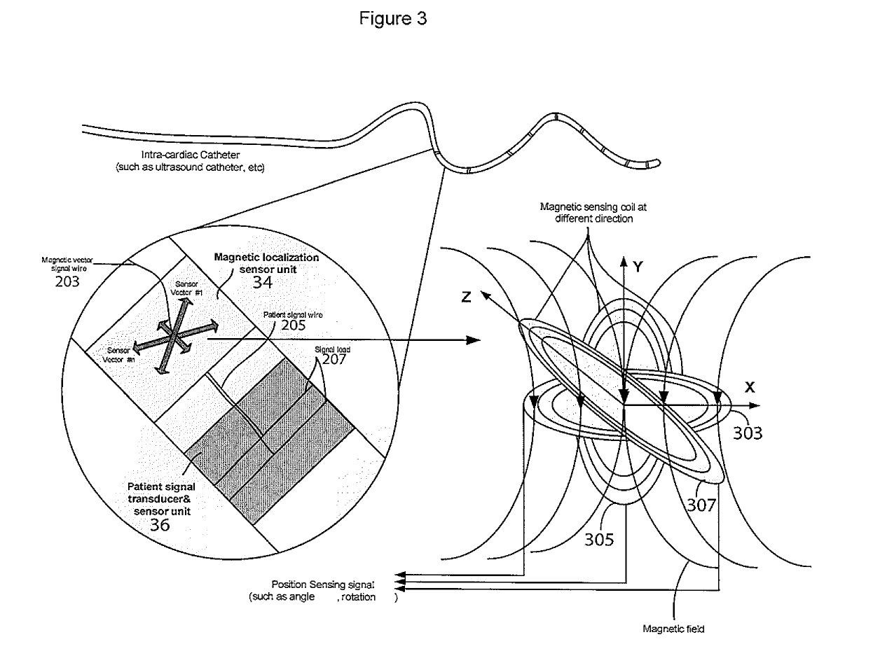

FIG. 3 shows a catheter localization sensor set including 3 coil magnetic sensors. Specifically, minimum localization sensors 34 comprise mutually orthogonal coils 303, 305 and 307 which intersect a magnetic field provided by field system 21 for determining X, Y and Z coordinate spatial location respectively. Magnetic coil sensors 303 and 305 are in the vertical plane (cross-sectional plane of the catheter) and magnetic coil sensor 307 is in the longitudinal direction (catheter direction). Different embodiments may employ different materials and configuration to capture direction and angular degree changes and may involve different electromagnetic converters. In operation magnetic copper coils 303, 305 and 307 oriented in different directions inside intra-cardiac catheter 41, cross the magnetic field (which is stable and substantially uniform) in different orientations which provides cross plane direction signal differences including cross-sectional area (square space) differences and angular differences, which results in different magnitude electrical signals. The position indicative signals are provided to electrical and treatment signal interface 47 and catheter position signals are extracted and used to track catheter rotation and angulation angle.

Signal processors 20 and 37 compare the strength of the generated electrical signals from the different coils and from the signal differences derive relative catheter twist angle and rotation angle. Catheter movement is small and slow which means a signal from a position localization sensor is relatively small. System 10 employs different magnetic field modes to track and characterize catheter position. For example, a dynamic (such as sinusoid) magnetic field is utilized to facilitate generation of a larger (more sensitive and reliable) signal from a sensor to derive position and movement data. Catheter position changes and magnetic field strength changes are also combined and used in catheter rotation and angle tracking. In other embodiments, additional coiled sensors are used to increase sensitivity, accuracy and stability of catheter segment rotation and angle tracking. In clinical application, catheter 41 may be twisted and rotated with different angles which requires detection of XYZ coordinate spatial position of a lead as well as direction of the lead (e.g., facing direction) for accurate ultrasound or ablation energy delivery, for example.

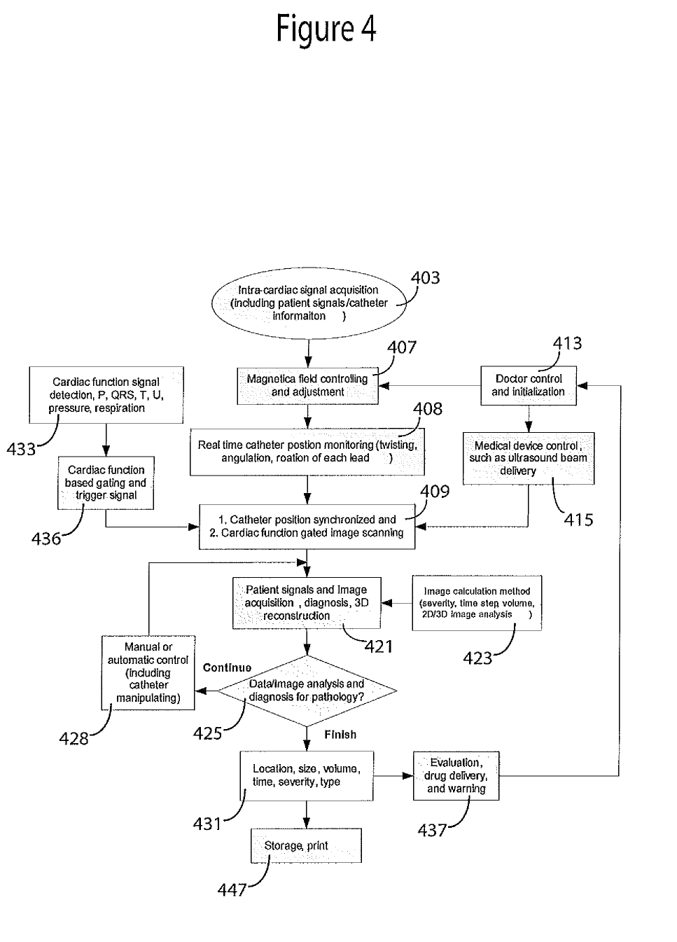

FIG. 4 shows a flowchart of a process employed by system 10 (FIG. 1) for intra-cardiac patient signal and image acquisition and scanning synchronized with catheter rotation and twisting. Catheter position and rotation information aids a user in identifying where to deliver energy in a cardiac chamber (tissue) e.g. for ablation. In an intra-cardiac ultrasound imaging application, catheter rotation and angulation affect ultrasound beam emission uniformity from a catheter and an emitted beam may be directional having a particular angle, such as 120 degrees. The catheter position and rotation information also supports synchronization and 3D image construction with less noise and artifacts.

System 10 in step 403 initiates intra-cardiac catheter insertion, twisting and rotation to get optimum patient signals at a desired position in a heart and in step 407 an external magnetic field provided by system 21 is initialized and adjusted in response to physician control in step 413. In step 408, system 10 tracks movement, rotation angle and twist of portions of the inserted catheter using set of magnetic sensors 34. Cardiac function gated image scanning is performed synchronized with catheter position in step 409 in response to device control provided in step 415 and a cardiac function based gating signal derived in step 436 from cardiac function signals including heart cycle segment representative signals (P wave, QRS wave, T wave, U wave) and signals identifying blood pressure and respiratory signals, for example, acquired in step 433. The gating signal is used for intra-cardiac image scanning to avoid noise and artifacts. The device control provided in step 415 controls ultrasound beam delivery in response to physician (or automatic) control in step 413.

In step 421, the catheter acquired image data and signals are extracted, processed and analyzed in real time to reconstruct a 3D imaging volume dataset and analyzed to provide a qualitative and quantitative diagnosis and characterization of abnormal cardiac functions and pathologies. In step 423 system 10 selects a process to use for analysis of acquired image and signal data to determine, medical condition, severity, time step used between image acquisition, chamber volume and to provide a 2D and 3D image reconstruction, for example. Selectable processes include a process for chamber edge determination for maximum chamber area and volume analysis and image registration for vessel and chamber analysis. In step 425 signal processor units 20 and 57 use a selected process to analyze an acquired image to determine image associated parameters and calculate image associated values and identify a particular medical condition by mapping determined parameters and calculated values to corresponding value ranges associated with medical conditions using predetermined mapping information stored in repository 17. The catheter acquired patient signals are analyzed in a region of interest (ROI) and associated with a heart cycle time stamp and related clinical events.

Steps 421 and 425 are iteratively repeated in response to manual or automatic direction and manipulation of the catheter in step 428, to identify medical condition characteristics from acquired catheter signals and image data. In response to completion of iterative image analysis of steps 421, 425 and 428, signal processor units 20 and 57 in step 431 determines location, size, volume, severity and type of medical condition as well as a time within a heart cycle associated with a medical condition. Signal processor units 20 and 57 initiate generation of an alert message for communication to a user in step 437 and provides medical information for use by a physician in making treatment decisions. The medical information includes pathology diagnosis, treatment delivery data (including catheter rotation and angulation) and any related warning. Reproduction device 19 presents images and signals acquired by a catheter to a user or a printer and stores images and signal data in repository 17 in step 447 and prompts a user with mapped treatment suggestions.

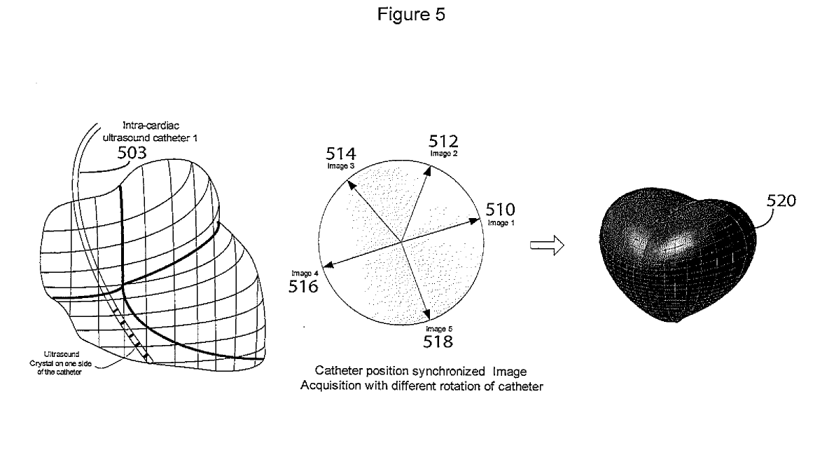

FIG. 5 illustrates intra-cardiac ultrasound catheter based 3D image scanning and reconstruction involving ultrasound catheter rotation tracking. The ultrasound catheter comprises multi-point crystals for sound generation and though flexible, is typically moved in nonlinear increments involving different degrees of twist and rotation with manual or step motor based control. Typically ultrasound crystals (used for ultrasonic beam generation and echo signal reception) are located on one side of a catheter and do not cover the full 360 degree circumference of a catheter. An intra-cardiac catheter of flexible material moves unevenly in different directions. Hence rotation and angulation distortion from catheter movement reduces resolution and precision of 3D image construction. System 10 (FIG. 1) synchronizes cardiac function signal gated 3D image acquisition for use in reconstruction to reduce distortion associated with use of non-synchronized image acquisition.

Catheter device 503 acquires 2D images 510, 512, 514, 516 and 518 at different positions and rotation angles in response to position and rotation angle synchronization data derived using set of magnetic sensors 34 and synchronized with cardiac function using an ECG signal. Ultrasound beam delivery is initiated in response to physician (or automatic) control. Signal processor units 20 and 57 process data representative of 2D images 510, 512, 514, 516 and 518 acquired at different rotation angles to provide 3D image construction 520. The 3D image volume reconstruction supports patient diagnosis and cardiac pathology severity tracking and characterization. Further, catheter rotation and position tracking improves efficiency of use of an ablation catheter delivering ablation energy in a particular direction or at a particular angle and not uniformly in a circle, for example. Ablation energy in a clinical application may otherwise be wasted by being mis-directed to blood and not human heart tissue.

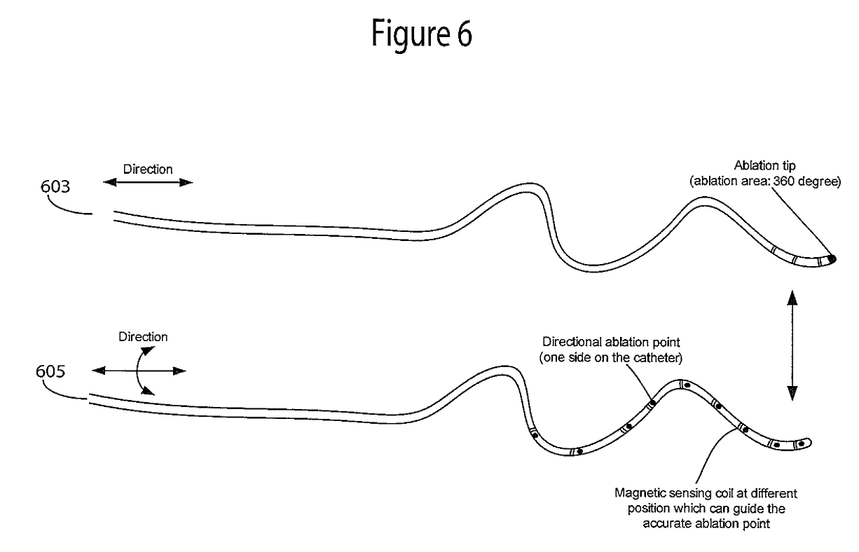

FIG. 6 shows different types of an ablation catheter. Specifically, catheter 603 is a known uni-point based ablation catheter delivering ablation energy uniformly around a 360 degree circumference in a clinical application. Catheter 605 is a multi-point directional ablation catheter using position, rotation angulation tracking and monitoring according to invention principles. Directional ablation catheter 605 advantageously reduces mis-application and waste of ablation energy by accurately localizing delivery of energy to fibrillation cardiac tissue at a focal area without mis-directing energy to incorrectly targeted tissue or blood. The catheter 605 system enables selection of ablation points and multi sequential point ablation reducing risk to patients.

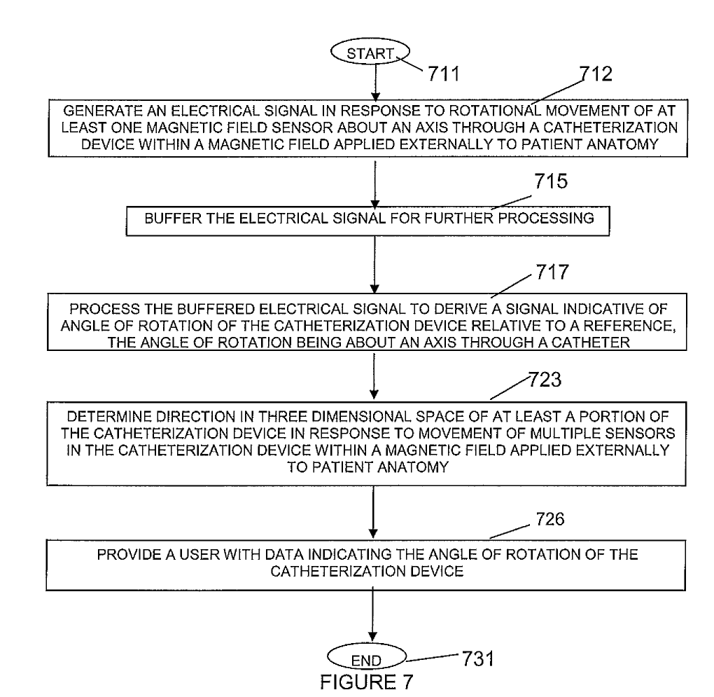

FIG. 7 shows a flowchart of a process used by interventional system 10 (FIG. 1) for internal anatomical examination. In step 712 following the start at step 711, at least one magnetic field sensor 34 generates an electrical signal in response to rotational movement of at least one magnetic field sensor 34 about an axis through catheterization device 41 within a magnetic field generated by system 21 applied externally to patient anatomy. The catheterization device includes at least one of, (a) an Ultrasound imaging unit and (b) an ablation function. At least one magnetic field sensor 34 is located in catheterization device 41 used for internal anatomical insertion. In one embodiment, at least one magnetic field sensor 34 comprises multiple sensors in substantially mutually orthogonal orientation for generating corresponding multiple electrical signals. The multiple of sensors (e.g., three sensors) are located in a tip of the catheterization device and the signal indicative of angle of rotation of the catheterization device indicates angle of rotation of the tip of the catheterization device. In step 715 signal interface 47 buffers the electrical signals for further processing.

Signal processor units 20 and 57 in step 717 process the buffered electrical signals to derive a signal indicative of angle of rotation the multiple sensors and catheterization device 41 relative to a reference, the angle of rotation being about an axis through catheter 41. The reference comprises a rotational angle of catheterization device 41 substantially at initial entry of the catheterization device into patient anatomy and is determined by the magnetic field applied externally to patient anatomy. The reference may comprise a rotational angle of another portion of catheterization device 41.

In one embodiment, catheterization device 41 includes multiple sets of sensors located in corresponding multiple different portions (including a tip portion) of the catheterization device. Signal processor units 20 and 57 process buffered electrical signals to derive a signal indicative of angle of rotation of individual portions of the catheterization device relative to the reference and process data indicative of angle of rotation of individual portions of the catheterization device to determine degree of twist of the catheterization device.

In step 723, directional data processor 27 determines direction in three dimensional space of at least a portion of catheterization device 41 in response to movement of multiple sensors 34 in catheterization device 41 within the magnetic field applied externally to patient anatomy. A spatial data processor in unit 20 determines three dimensional spatial location of at least a portion of the catheterization device in response to movement of multiple sensors in the catheterization device within the magnetic field applied externally. In step 726, system 10 provides a user with data indicating the angle of rotation of catheterization device 41 via reproduction devices such as displays 19 and 39, for example. The process of FIG. 7 terminates at step 731.

A processor as used herein is a computer, processing device, logic array or other device for executing machine-readable instructions stored on a computer readable medium, for performing tasks and may comprise any one or combination of, hardware and firmware. A processor may also comprise memory storing machine-readable instructions executable for performing tasks. A processor acts upon information by manipulating, analyzing, modifying, converting or transmitting information for use by an executable procedure or an information device, and/or by routing the information to an output device. A processor may use or comprise the capabilities of a controller or microprocessor, for example, and is conditioned using executable instructions to perform special purpose functions not performed by a general purpose computer. A processor may be coupled (electrically and/or as comprising executable components) with any other processor enabling interaction and/or communication there-between. A display processor or generator is a known element comprising electronic circuitry or software or a combination of both for generating display images or portions thereof.

An executable application, as used herein, comprises code or machine readable instructions for conditioning the processor to implement predetermined functions, such as those of an operating system, a context data acquisition system or other information processing system, for example, in response to user command or input. An executable procedure is a segment of code or machine readable instruction, sub-routine, or other distinct section of code or portion of an executable application for performing one or more particular processes. These processes may include receiving input data and/or parameters, performing operations on received input data and/or performing functions in response to received input parameters, and providing resulting output data and/or parameters. A user interface (UI), as used herein, comprises one or more display images, generated by a display processor and enabling user interaction with a processor or other device and associated data acquisition and processing functions.

The UI also includes an executable procedure or executable application. The executable procedure or executable application conditions the display processor to generate signals representing the UI display images. These signals are supplied to a display device which displays the image for viewing by the user. The executable procedure or executable application further receives signals from user input devices, such as a keyboard, mouse, light pen, touch screen or any other means allowing a user to provide data to a processor. The processor, under control of an executable procedure or executable application, manipulates the UT display images in response to signals received from the input devices. In this way, the user interacts with the display image using the input devices, enabling user interaction with the processor or other device. The functions and process steps herein may be performed automatically or wholly or partially in response to user command. An activity (including a step) performed automatically is performed in response to executable instruction or device operation without user direct initiation of the activity.

The system and processes of FIGS. 1-7 are not exclusive. Other systems, processes and menus may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention. The system provides continuous cardiac imaging by tracking position, rotation and twist of one or more portions of a catheter. Further, the processes and applications may, in alternative embodiments, be located on one or more (e.g., distributed) processing devices on a network linking the units of FIG. 1. Any of the functions and steps provided in FIGS. 1-7 may be implemented in hardware, software or a combination of both.

What is claimed is:

1. An interventional system for internal anatomical examination, comprising:
a catheterization device for internal anatomical insertion including,
at least one magnetic field sensor for generating an electrical signal in response to rotational movement of said at least one magnetic field sensor about an axis through said catheterization device within a magnetic field applied externally to patient anatomy and
a signal interface for buffering said electrical signal for further processing;
a signal processor for processing the buffered electrical signal to derive a signal indicative of angle of rotation of said catheterization device relative to a reference, said angle of rotation being about an axis through a catheter; and
a reproduction device for presenting a user with data indicating said angle of rotation of said catheterization device.

2. A system according to claim 1, wherein
said at least one magnetic field sensor comprises a plurality of sensors in substantially mutually orthogonal orientation for generating a corresponding plurality of electrical signals,
said signal interface buffers said electrical signals for further processing and
said signal processor processes the buffered electrical signals to derive a signal indicative of angle of rotation of said catheterization device.

3. A system according to claim 2, wherein
said plurality of sensors are located in a tip of said catheterization device and said signal indicative of angle of rotation of said catheterization device indicates angle of rotation of said tip of said catheterization device.

4. A system according to claim 2, wherein
said plurality of sensors comprises three sensors.

5. A system according to claim 2, wherein
said signal processor processes the buffered electrical signals to derive a signal indicative of angle of rotation of said plurality of sensors.

6. A system according to claim 2, wherein
said reference comprises a rotational angle of said catheterization device substantially at initial entry of said catheterization device into patient anatomy.

7. A system according to claim 2, wherein
said reference is determined by said magnetic field applied externally to patient anatomy.

8. A system according to claim 2, wherein
said reference comprises a rotational angle of another portion of said catheterization device.

9. An interventional system for internal anatomical examination, comprising:
a catheterization device for internal anatomical insertion including,
a plurality of sensors in substantially mutually orthogonal orientation for generating electrical signals in response to rotational movement of said plurality of sensors about an axis through said catheterization device within a magnetic field applied externally to patient anatomy and
a signal interface for buffering said electrical signals for further processing;
a signal processor for processing the buffered electrical signals to derive a signal indicative of angle of rotation of said catheterization device relative to a reference, said angle of rotation being about an axis through said catheterization device; and
a reproduction device for presenting a user with data indicating said angle of rotation of said catheterization device.

10. A system according to claim 9, wherein
said catheterization device includes a plurality of sets of sensors located in a corresponding plurality of different portions of said catheterization device and
said signal processor processes buffered electrical signals to derive a signal indicative of angle of rotation of individual portions of said catheterization device relative to a reference.

11. A system according to claim 10, wherein
said signal processor processes data indicative of angle of rotation of individual portions of said catheterization device to determine degree of twist of said catheterization device.

12. A system according to claim 10, wherein
said plurality of different portions of said catheterization device include a tip portion.

13. A system according to claim 9, including
a spatial data processor for determining three dimensional spatial location of at least a portion of said catheterization device in response to movement of a plurality of sensors in said catheterization device within a magnetic field applied externally to patient anatomy.

14. A system according to claim 13, including
a directional data processor for determining direction in three dimensional space of at least a portion of said catheterization device in response to movement of a plurality of sensors in said catheterization device within a magnetic field applied externally to patient anatomy.

15. A system according to claim 9, wherein
said catheterization device includes at least one of, (a) an Ultrasound imaging unit and (b) an ablation function.

16. A method for providing interventional system position data for internal anatomical examination, comprising the activities of:
generating an electrical signal in response to rotational movement of at least one magnetic field sensor about an axis through a catheterization device within a magnetic field applied externally to patient anatomy, said at least one magnetic field sensor being located in said catheterization device usable for internal anatomical insertion and
buffering said electrical signal for further processing;
processing the buffered electrical signal to derive a signal indicative of angle of rotation of said catheterization device relative to a reference, said angle of rotation being about an axis through a catheter; and
providing a user with data indicating said angle of rotation of said catheterization device.

17. A method according to claim 16, wherein
said at least one magnetic field sensor comprises a plurality of sensors in substantially mutually orthogonal orientation for generating a corresponding plurality of electrical signals and including the activities of,
buffering said electrical signals for further processing and
processing the buffered electrical signals to derive a signal indicative of angle of rotation of said catheterization device.
